(19) United States
(12) Patent Application Publication
(10) Pub. No.: US 2007/0294109 A1
(43) Pub. Date: Dec. 20, 2007

(54) Method and system for creation of an integrated medical record via a communications computer network

(76) Inventor: John B. Costello, St. Louis, MO (US)

Correspondence Address: Paul M. Denk, Suite 170, 763 S. New Ballas Road, St. Louis, MO, 63141, US

(21) Appl. No.: 11/471,419

(22) Filed: Jun. 20, 2006

(51) Int. Cl. G06F 19/00; A61B 5/00

(52) U.S. Cl. 705/3; 600/300

(57) ABSTRACT

A method and system for single form creation of a patient visit medical record via the Internet that integrates with the financial system of the healthcare provider. A patient record generally has medical data and financial data. This system improves processing medical data, patient care, and financial information. First, the system provides a digital visit form, hosted on a server, and completed at the user's location. Second, the digital form contains lists, boxes, and buttons for data entry and work flow. Third, the system joins the diagnosis and the plan of the medical record including codes for the visit CPT, the diagnosis, procedure, and labs, prescription, pharmacy, referred provider, notes-and next appointment. This medical data then feeds into billing. Fourth, upon selecting the SAVE button, the system sends the medical and financial data to the server that updates the medical record, initiates medical and financial processes, and updates queue.

### CROSS REFERENCE TO RELATED APPLICATION

This application depends from provisional application having Ser. No. 60/199,412, filed on Apr. 24, 2000 and Ser. No. 09/837,895 filed on Apr. 18, 2001.

### STATEMENT REGARDING FEDERALLY SPONSORED RESEARCH OR DEVELOPMENT

N/A

### TECHNICAL FIELD

The present invention relates to a computer method and system over a network for creating a medical visit note in a single elongated template driven form with strategic use of DDLs, LBs, and buttons, to maximize the efficiency of the workflow and by integrating the required billing information at the end of the medical visit note, data which is necessary for the business requirements of the healthcare provider further enhancing workflow efficiency.

### BACKGROUND OF THE INVENTION

To better understand the concept of this invention, an understanding of the routine procedure, various words, medical terms, medical business practices and processes, business terms and entities used by the medical practitioner in processing of patients, will be required.

The process of clinical medicine consists of at least these minimum steps; 1) the patient is scheduled for a visit, 2) the patient's clinical visit which include components, 2a) history taking and physical exam and 2b) diagnosis, testing and treatment planning and 3) sign out, which includes, 3a) setting up the next visit, 3b) scheduling studies or referrals.

The next process is the financial steps of a clinical visit. They are as follows: 1) the patients insurance information is gathered and verified, 2) the patient is seen and a charge sheet records the charges for that visit; which is used for charge entry into some form of billing system; 3) the data goes to the patient's ledger, a listing of his charges and payments, and then the statements to the patients are generated from this ledger. 4) The data from the visit is forwarded, now usually electronically, by point-to-point services to insurance companies for processing the claims for payment to the physician for the service. There is also usually a component for the patient in arrears for collections.

All processes follow the regulations required by the various governing bodies such as Health Care Financing Administration The next definition of importance would be a summary of the components that are in a Medical Record. These include: 1) a charge sheet or Super bill which are often used as synonyms; 2) the demographic, insurance and referral information; 3) a face sheet, which includes diagnoses, allergies, surgeries etc.; 4) a medication list; 5) a clinical note with vital signs, memos and procedure notes; 6) a laboratory section, either created in house or outside; 7) a radiology section, either created in house or outside; 8) special tests, either created in house or outside, including pathology or biopsy reports; 9) outbound letters; 10) inbound letters, including other physician's notes, hospital discharge summaries and op notes; 11) insurance papers; 12) consents signed by patient.

The next definition is the Electronic Medical record. The electronic medical record is all of the above components being created and stored on a computer or some form of electronic device.

The next definition would be a listing and defining of the systems on which an electronic record could reside. There are three principle forms of electronic medical charts. 1) The single user, one computer that stores both the data and the software to display and manipulate the data. 2) The server client systems where the data is stored by the server and the clients have the software to display and manipulate the data. The key-defining element of these systems is that the design of hardware and software is closed with no outside access even though the software and network can talk to multiple users and locations. The third form of system on which medical records are hosted would be the Internet. This form of system allows universal access with proper user authentication.

The Internet comprises a vast number of computers and computer networks that are interconnected through communication links. The interconnected computers exchange information using various services, such as electronic mail, Gopher, and the World Wide Web (“WWW”). The WWW service allows a server computer system (i.e., Web server or Web site) to send graphical Web pages of information to a remote client computer system. The remote client computer system can then display the Web pages. Each resource (e.g., computer of Web page) of the WWW is uniquely identifiable by a Uniform Resource Locator (“URL”). To view a specific Web page, a client computer system specifies the URL for that Web page in a request (e.g., a Hypertext Transfer Protocol (“HTTP”) request). The request is forwarded to the Web server that supports that Web page. When that Web server receives the request, it sends that Web page to the client computer system. When the client computer system receives that Web page, it typically displays the Web page using a browser. A browser is a special-purpose application program that effects the requesting of Web pages and the displaying of Web pages.

Currently, Web pages are typically defined using Hypertext Markup Language (“HTML”). HTML provides a standard set of tags that define how a Web page is to be displayed. When a user indicates to the browser to display a Web page, the browser sends a request to the server computer system to transfer to the client computer system an HTML document that defines the Web page. When the requested HTML document is received by the client computer system, the browser displays the Web page as defined by the HTML document. The HTML document contains various tags that control the displaying of text, graphics, controls, and other features. The HTML document may contain URLs of other Web pages available on that server computer system or other server computer systems.

Of all human endeavors, the World Wide Web is especially conducive to managing both the medical and business needs of health care providers. The best patient care results from the most accurate, timely, open and cooperative flow of information. Ideally, the goal of any invention to improve the work processes of the clinical and business aspects of Medicine would be to provide all the medical and business functionality required in healthcare provider's office other than “hands-on” diagnostic and therapeutic patient procedures in one tool. The Online Medical Record is the key piece of software to create the efficient information flow embodied in this patent.

Physician acceptance of electronic records has been extremely slow because of the bad workflow of prior systems to document patient visits. Given the number of tasks the provider is required to do in a day's time, the healthcare provider will not automate their current workflow unless it is medically, financially and business process critical. Inefficiency was due to at least two components. First, the physician's interaction with an electronic record was too slow and cumbersome for them to want to use it. Second, up to now, most medical records were independent of other components required in a physician's office, those other components as defined above, their business needs and the patient's information needs.

Further, costly software licenses and servers systems have limited the adoption by small practices (<5 person groups) comprising 70% of the physician market. Licenses often only provide functionality in building blocks with the overall price growing as systems grow in capability. Once systems are implemented, they often require costly maintenance and upgrades. Finding talented IT staff continues to be a more difficult problem. Purchase and use of present systems often means the database is not accessible or easily portable to other systems or users outside the local network. Patient medical records, Patient Data, and the business data of the practice, Practice Data, come under the propriety control of the software vendor.

Some software vendors have moved software applications from the individual office to the Web and have provided access to the application as a service. These ASP providers add value to the healthcare provider by delivering more cost effective access. However, the efficiencies of linking up all aspects of a physician's medical and business needs in at the crucial point of care has just not been accomplished because the software until now to accomplish this task and the efficiency of design of business processing which we are trying to patent has never been proposed.

In this Age, the central transaction in the practice of medicine is the person-to-person interaction between the healthcare provider and the patient. If this interaction can be cleanly and efficiently recorded, then errors and workload will be reduced and the provider can attain better patient care. The Online Medical Record (OMR), containing Patient and Business Data, created via editing an HTML document, and transmitted over the Internet, is the embodiment of this patent.

See U.S. Pat. No. 5,065,315 for a system and method for scheduling and reporting patient related services including prioritizing services in a hospital. The United States patent to Feinberg, U.S. Pat. No. 6,082,776, simply pertains to the capturing of data, medical or otherwise, in a form that is compressed so as to save space and time in whatever medium the data is stored. Essentially, it appears that the data is bar coded, and has no bearing or relevancy relating to the subject matter of Applicant's invention. Applicant's invention provides a single page compilation and storage of data relating to medical information pertaining to a specific patient, at the point of service.

### SUMMARY OF THE INVENTION

The embodiment of the present invention provides a method and system for single page/form creation of an Online Medical Record which simultaneously integrates the medical care provided and recorded during a patient visit with the financial requirements of the provider for that particular patient visit with strategic use of devices (DDLs, LBs, Note boxes, Profile buttons, and Search buttons) which maximize workflow efficiency and culminates with a single selection Save button that initiates further processing of all the data by the server. This is done by the presentation of an HTML page that contains all the requisite data fields and devices that cover the medical and financial side of a patient visit. These fields contain data that is used by both those skilled in the medical arts and those skilled in the accounting arts. Upon the single selection of the Save button, the system automatically updates queues for further processing of all business transactions. The single HTML page, when completed, then allows the provider to select the medical business and financial transactions, including without limitation, to payment, claims processing, recording laboratory data, pharmacy transactions, referrals, procedures, treatments given and prescribed, and many of the other medical and financial transactions that are performed by the physician, and his/her administrative staff when operating a small medical office, but actually a medical office of any size, incorporating and utilizing the system of this invention.

This invention contemplates the method and system for creation of an integrated medical patient's record via a computer, or a communications computer network, which includes the provision of a record of standard patient personal and medical history, and which is capable of receiving data pertaining to a specific visit for the patient being treated, providing means for inputting data relative to the personal history of the specific patient to be examined and treated, inputting data relative to the specific medical information determined during that examination and visit for the patient, providing a record of the patient's diagnosis, studies and treatment during that particular visit, initially inputting data relative to the patient's diagnostic studies, treatment and visit for that specific visit, and then inputting the financial requirements allowed for medical coverage by the patient's insurance provider and the patient, then either simultaneously or consecutively deriving and calculating the financial obligations regarding the patient's said studies, treatment and visit, for determining the patient and the insurance providers financial obligations, providing a single page record, upon the pressing of the “Save” key upon the computer, for said diagnosis, studies, treatment, and examination, and the financial obligations of the parties involved, and then storing electronically in memory the statement of the patient's medical history for that visit, in addition to the patient's previously determined medical history, in addition to preserving the financial obligations of the patient, and provider for that specific patient visit. In addition, the system includes the method for inputting data relative to the physician's visit code that categorizes the charges made relative to the type of diagnostic study, treatment, and diagnosis provided by the physician for that particular visit. Furthermore, the determination of the financial requirements of the allowed medical coverage for the patient's visit is created in a single step by the computer system and its software herein.

### BRIEF DESCRIPTION OF THE DRAWINGS

FIG. 1 illustrates a simplified embodiment of the single HTML page visit form with the integration of the medical and financial portions of a medical visit;

FIG. 2 illustrates a more explicit embodiment of the visit form;

FIG. 3 illustrates the embodiment of the strategically placed DDLs, LBs, and Search buttons which accelerate the workflow efficiently;

FIG. 4 is a flow diagram of data entry via: selection of topics; searching for topics, if necessary; use of the topic profile, if desired; and entry/edit of data elements;

FIG. 5 is a flow diagram of the post-Save button selection processes and queues which operate on data collected on the visit form; and

FIG. 6 illustrates the configuration of data elements.

APPENDIX A furnishes the process software code for the system of this invention;

APPENDIX B is a disclosure of the actual visit form as displayed on the computer screen for a patient;

APPENDIX C is the form displayed on the computer screen showing the computation and charges for the previous treatment;

APPENDIX D is an example of the stored form for a particular visit after the SAVE button selection; and

APPENDIX E is identified as a clinical summary for patient.

### DETAILED DESCRIPTION OF THE INVENTION

The presented invention provides a method and system for creating and recording on a single form the medical and financial data of a medical visit in a client/server environment. The single form, joining of the medical and financial data, strategic use of devices such as, DDLs, LBs, Text entry boxes, Profile buttons, and Search buttons, and the single Save button selection, reduce the number of user interactions needed to record a medical visit and the charges incurred. This form is presented by the server over a network, such as the Internet, for the user through a client system to place specific information in each field of the form as the patient visit dictates. When the data set for that visit is complete a single Save button on that form instructs the server to save all data pertaining to the visit. The medical data is sent to required subroutines outside of the medical visit form for recording and further use for non-billing purposes as a medical data set. The financial data is sent to required subroutines outside of the medical visit form for recording and further use for billing purposes. The components required in a medical office to run the business side of the office are an accounting system (ledger) for tracking patients monies owed to the practice, an electronic filing component to file claims to the insurance companies, and some form of patient medical visit recording which for most offices is still a paper record. The speed and efficiency of recording visit data, the ease of workflow, and the minimum number of clicks and entries required to record the maximum data (without inventing data unscrupulously) is one of keys to the value of this invention. The other key is that all prior medical practice management systems have required double entry. The Diagnosis, studies and treatment went on the paper or electronic medical chart and the procedure code which described the visit and in house testing that the office charged the patient went on a paper superbill. A second step was required to record this data into the practice management computer system used by the office to file insurance claims for payment or to bill the patients. Some offices are not yet doing computers and do this second step is on paper.

To present more clearly in this detailed description how the workflow is made more efficient, it is important to briefly touch on the Configuration section of our invention. Under the Configuration section inside the program, data elements for the DDLs and LBs are populated at the start of use of the program. These are HTML devices which present a list of possible choices. When the user sees these fields on the client, the user is limited to those preset up choices.

### DESCRIPTION OF THE PREFERRED EMBODIMENT

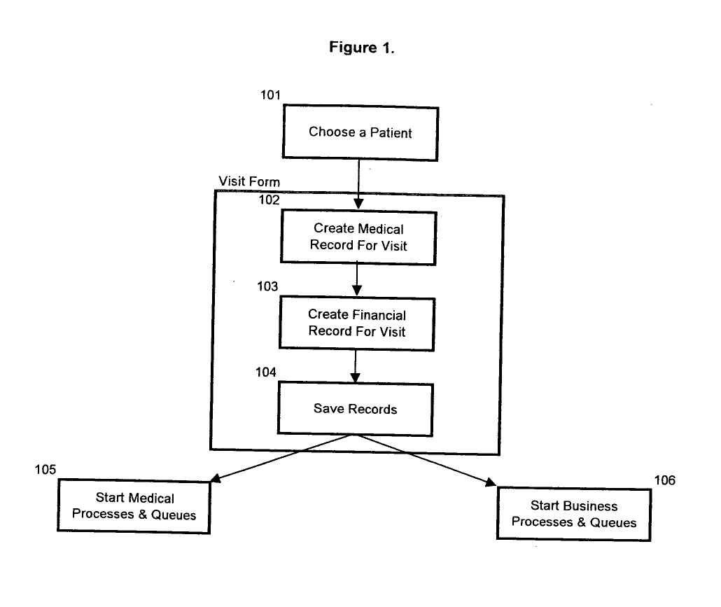

FIG. 1 illustrates a simplified embodiment of the single HTML page visit form with the integration of the medical and financial portions of a medical visit. This figure contains: the ‘choose a patient’, section 101; a process for creating medical visit information, section 102; a process for creating financial visit information, section 103; a single push Save button, section 104; a function for starting medical processes and queues, section 105; and a function for starting business processes and queues, section 106. The process flow is as follows: section 101 flows to section 102; section 102 flows to section 103; section 103 flows to section 104; and section 104 flows to both section 105 and section 106. One skilled in the art would appreciate that these various sections can be modified for different medical or surgical specialties. Further detail is presented in the next figure.

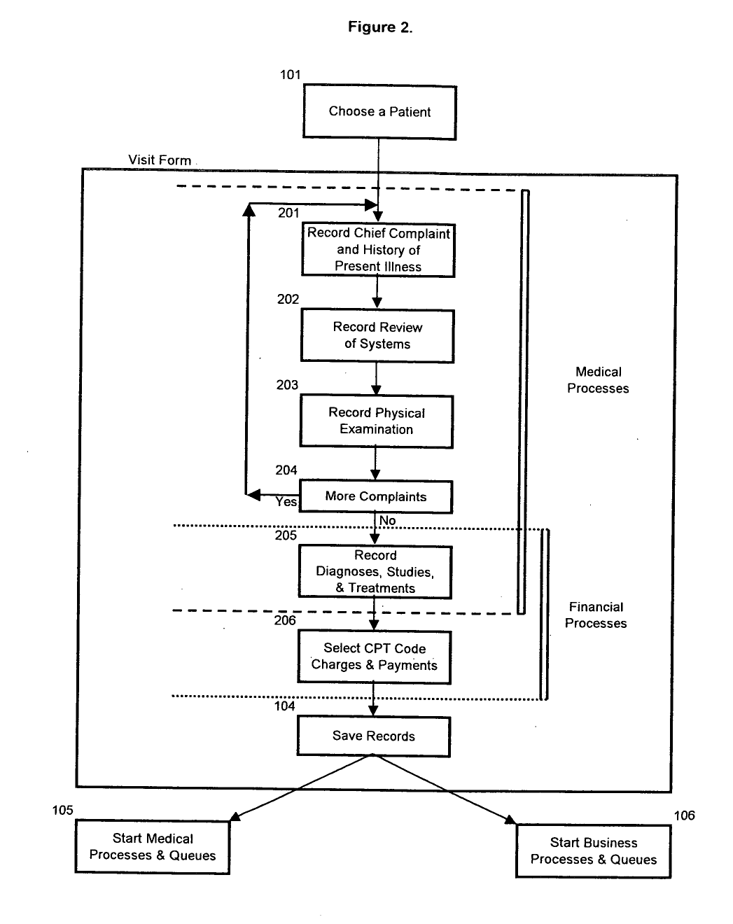

FIG. 2 illustrates a more detailed schematic of the steps embodied in this invention. This figure contains: the ‘choose a patient’, section 101; a process for recording the Chief Complaint and History of the Present Illness, section 201; a process for recording the Review of Systems, section 202; a process for recording the Physical Examination, section 203; a decision box asking if there are more complaints to record, section 204; a process for recording Diagnosis, Studies Ordered and Treatments, section 205; a process for CPT Code selection, Charges, and Payments, section 206; a single push Save button, section 104; a function for starting medical processes and queues, section 105; and a function for starting business processes and queues, section 106. It now becomes clearer where the data entries that satisfy the medical and business side of a medical visit over lap.

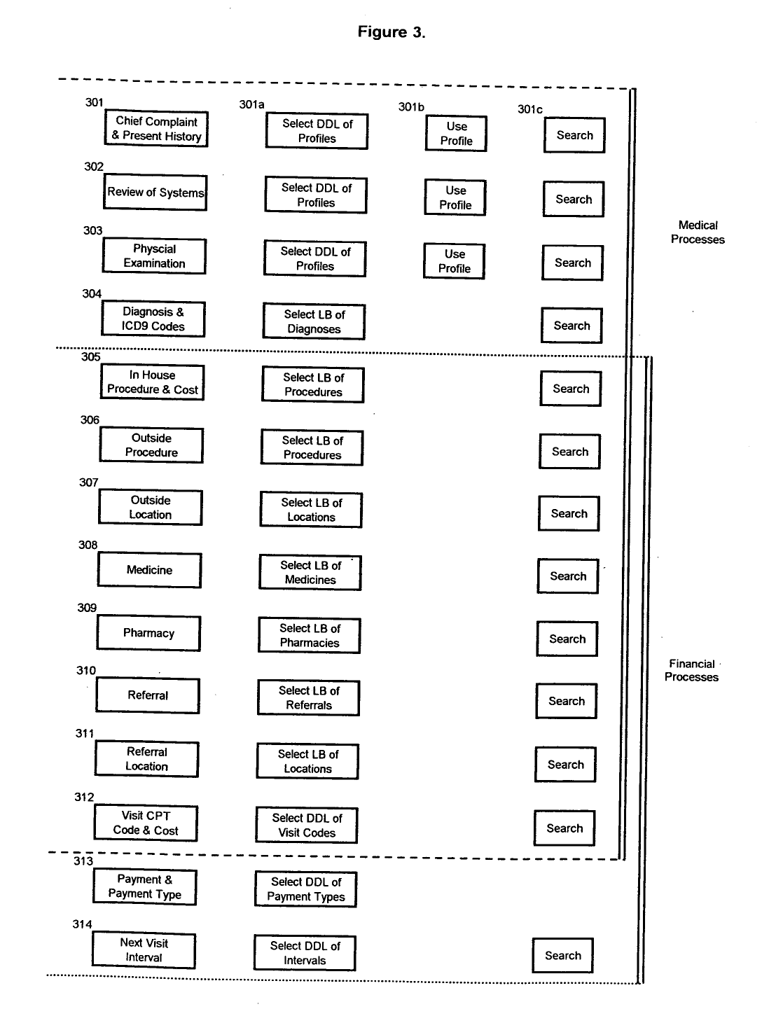

FIG. 3 illustrates the component sections of the Medical Processes and Financial Processes illustrated within the Visit Form on FIG. 2. This shows the use of the DDLs, LBs, Profile buttons, and Search buttons to provide the efficient workflow. Combinations of these component sections will depend on the type of medical practice desired and can be used in various environments other than over data networks or the Internet. This figure contains: a Chief Complaint/Present History process, section 301; a Review of Systems process, section 302; a Physical Examination process, section 303; a Diagnosis & ICD9 Codes process, section 304; an In house tests/procedures & cost process, section 305; a outside office tests/procedures process, section 306; an outside office location process, section 307; a prescription medication process, section 308; a prescription medication location process, section 309; a Referral to other specialist process, section 310; a Referral to other specialist location process, section 311; a visit procedure code process, section 312; a payment and payment type process, section 313; and a Next visit interval process, section 314. The usual workflow is to step through these sections. Strategically placed in many of the sections, there will be profile/template driven entry of data elements in boxes presented under each section. This is the function of the DDL box. It provides a pick list box of pre-configured topics to populate the fields pertinent to that component section. When the complexity or quantity of choices in a pick list box becomes too burdensome to remember, the use of a strategically placed Search button allows fast sorting and recall of the data set that is desired for that component section. This is illustrated by the Select DDL of Profiles, subsection 301a; the Use Profile, subsection 301b ; and the Search button, subsection 301c. The steps through this are straightforward. First, look at the subsection 301a. If it is not easy to find the item then choose subsection 301c, input the desired key word and when the result is presented and selected, you will return to subsection 310a and with the item selected. Then the subsection 301b can be activated to bring in the profile/full data set that has been stored in the configuration section on the server. This process is repeated for 302 through to 314.

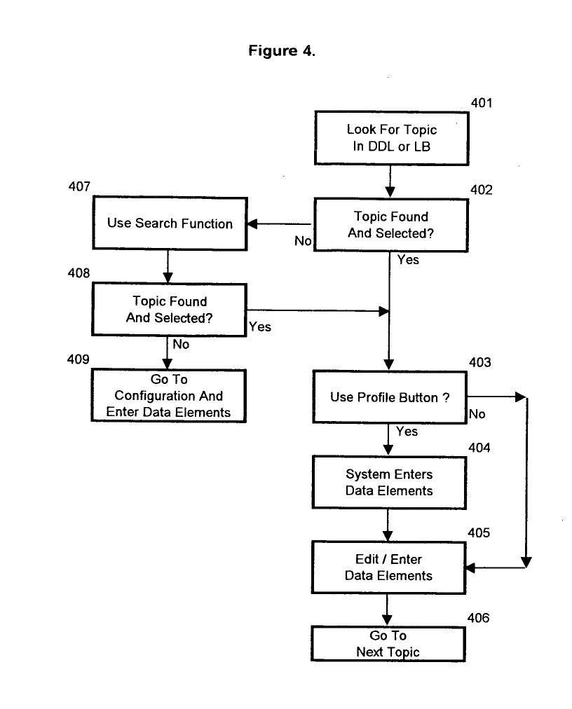

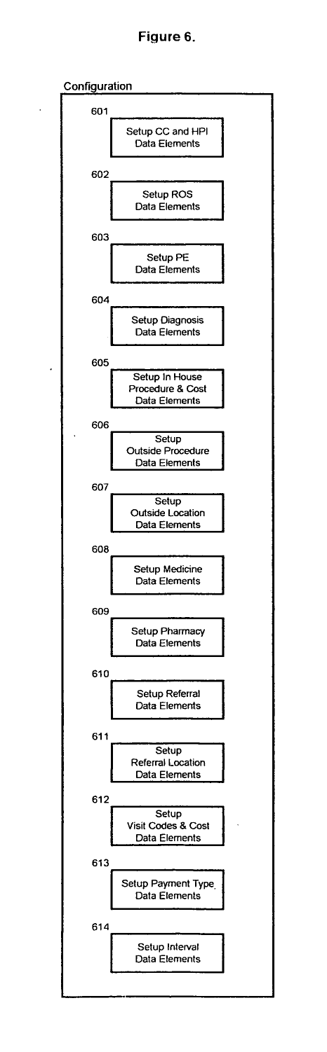

FIG. 4 illustrates the flow among the FIG. 3 subsections within the component sections. This describes the flow of data entry via: selection of topics; searching for topics, if necessary; use of the topic profile, if desired; and entry/edit of data elements. This figure contains: a process Look For Topic in DDL or LB, section 401; the decision box Topic Found and Selected asking if the topic was found in the DDL or LB, section 402; a decision box Use Profile Button asking if the user has selected the Profile button, section 403; a System Enters Data Elements process wherein the system loads data elements that have been stored in the configuration for that Profile, section 404; a Edit/Enter Data Elements process, section 405; a Go To Next Topic step, section 406; a Use Search Function process, section 407; a decision box Topic Found and Selected asking if the topic was found by the Search Function, section 408; and a Go To Configuration And Enter Data Elements step, section 409. The process flow is as follows: Section 401 flows to Section 402. If the answer to section 402 is “Yes”, then the process goes to section 403. If the answer to section 402 is “No”, then the process flows to section 407. Section 407 flows to section 408. If the answer to section 408 is “Yes”, then the process flows to section 403. If the answer to section 408 is “No”, then the process flows to section 409. Section 409 flows to the Configuration, FIG. 6. If the answer to section 403 is “Yes”, then the process goes to section 404. Section 404 flows to section 405. If the answer to section 403 is “No”, then the process goes to section 405. Section 405 flows to section 406.

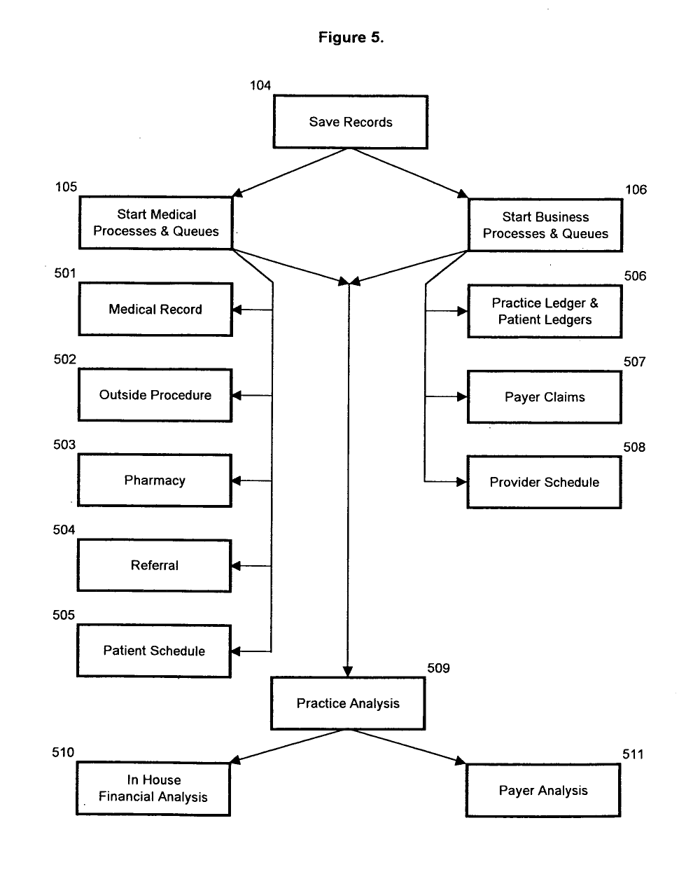

FIG. 5 Illustrates the uses of the data on the visit form and then saved by the single Save button push at the close of the visit form. It contains: the Save Record button, section 104; a Update Medical Processes and Queues process, section 105; a Update Business Processes and Queues process, section 106; a Medical Records process, section 501; a Outside Procedure process, section 502; a Pharmacy process, section 503; a Referral section 504, a schedule section 505, a Practice and patient ledgers section 506, a Payer claims segment 507, a business schedule section 508, a practice analysis section 509, a in house financial analysis section 510 and a Payer analysis section 511. One skilled in the art would appreciate that this list of medical and business queues and processes is by no means complete and that it will vary based on the specialty of Medicine and the business form of the practice, i.e. if an independent practice or if it is owned by a corporation.

To clarify the meaning of each process and queues is as follows. The medical record stored is the data for the patient for one visit. The outside procedures data are those diagnostic and therapeutic procedures ordered for that patient during and as a consequence of that visit. The pharmacy data is that data that gets a prescription for the patient. The Referral data is that data that gets a referral to another Provider for that patient. The Patient schedule gets the patient scheduled for their next visit with the provider. The Business queue components start with the patient ledger which adds charges and payments for the individual patients accounts receivable. The practice ledger is updated also with each individuals charges and payments. The payer claims is the electronic transfer of data as required by the federal government for adjudication and payment by third parties for a patients visit. The business schedule is updated to tell the provider when and where they are needed for care of the patient, i.e. if the patient needs surgery, the providers schedule blocks out time for this required work. The practice analysis is the grouping and comparing of medical or business activities to determine if time used is commensurate with reimbursement.

FIG. 6 illustrates the some of component parts of the Configuration section that reside on the server. It has a Set up Chief complaint and history of the present illness responses section 601, a Set up Review of systems responses section 602, a Set up Physical exam responses section 603, a Set up Diagnosis section 604, etc. The purpose of these sections is to create in the background on the server the data sets used in the foreground by the user when the user is recording a patient visit on the visit form.

APPENDIX A provides a display of the process software code for generating the display of the various forms upon the computer screen, to allow for filling in of medical and financial data relating thereto, with respect to the particular patient being treated for that visit, and which then computes the financial data relating to the cost of that visit, for billing purposes either to the patient, or to the insurance provider, transmits such data, and then stores all that data relative to that patient. This source code provides a full listing of the steps conducted when performing the method under the system of this invention, for integrating entirely the medical records of a particular patient, and then transmit such data either into the computer for storage, or transmit the same through a communications network to the various related providers. The end result is that the medical services are completely documented, including all treatment, laboratory tests, diagnostic studies, etc., and in addition, the business aspects of the overall medical treatment, such as claims processing, determining insurance coverage, etc., is all documented in a single simultaneous, or concurrent or consecutive process.

APPENDIX B is an example of a visit form used by a practitioner administering an internal medicine office. One skilled in the art realizes that variations on this visit form may occur depending upon the type of medicine, surgery, podiatric, chiropractic, Physical Therapy, Occupational Medicine, or any other care giver patient interaction that may be performed. It is these forms that may be brought up by the source code, on the computer screen, the data filled in, to provide for a complete record of a patient's visit.

APPENDIX C is a continuation of the visit form as shown and described in APPENDIX B, but what APPENDIX C shows is the clinical side of the patient's visit so that after the visit form has been filled in, in APPENDIX B, and in APPENDIX C, APPENDIX C provides the continuation of the processing of the data relating to the patient, and also adds, without further reentry or manual participation, the calculation of the business aspects of the particular visit. But, in addition, once the “Save” button is initiated upon the computer, that financial data is then processed with the patient's previous financial history relative to treatment by the practitioner, and calculates the balance due to the attending physician. Furthermore, it is possible, through the use of this program, that the financial data so calculated will then be transmitted to the insurance provider, and elsewhere, for processing of the practitioner's claim for financial reimbursement for that particular visit.

APPENDIX D illustrates the saved version of the patient's visit. Obviously, there are many other items of data that flow to other processes and queues of this program, as previously enumerated, and as processed by the source code, that are not represented in this particular display of a single patient's visit, as shown in the screen display of this Appendix.

APPENDIX E shows a clinical summary for the individual patient, and which is embodied into the single page record for the patient.

As an example of the versatility of the usage of the method and system of this invention, it is just as likely that instead of providing a single medical records sheet for the patient to fill in, and then inputting that data through the computer keyboard into the system to provide specific patient data, it may be, for example, that such a completed record may simply be scanned, by a scanner, for inputting into the computer, to provide that inputting of data relative to the specific patient in attendance for the visit. In addition, it is just as likely the computer network communication system may include the transmission, over dedicated lines, from the physician's office directly to the insurance provider informing the latter of the examination, treatment, for the visit, and the provider's financial obligations under its policy to compensate for the treatment of the insured. These are examples as to how this system can be integrated into an overall network, to substantially reduce the paperwork required to conduct a medical office, and reduce it significantly to a process that is handled by a single computerized software, freeing the physician and his staff from the normally substantial paperwork involved in providing medical treatment in the office, and any patient visits made at the hospital.

Variations or modifications to the subject matter of this invention may occur to those skilled in the art upon review of the invention as described herein, as depicted in its drawings. Further variations may be considered by those skilled in the art upon review of the summary herein, and upon undertaking a study of the description of its preferred embodiment, in view of the drawings, and the Appendices included herewith.

1. A novel and unique method and system for creation while in the presence of a patient, including an integrated single page medical patient's record via a communications computer network, including:
providing a record of standard patient personal and medical history all on said single page and capable of receiving data pertaining to a specific visit for the patient being treated;
inputting data relative to the personal history of the specific patient all on said single page to be examined and treated;
inputting data relative to specific medical information determined during the specific patient's examination and visit on the same single page;
providing a record of the patient's diagnosis, studies and treatment all on said single page during this specific patient visit;
calculating the financial obligations for the specific patient's examination and visit on said single page;
providing said single page record for the diagnosis, studies ordered, and treatment for that patient's specific visit;
providing on the same single page record a statement of the patient's and provider's financial obligations for the examination and visit; and
storing in memory the statement of the patient's medical history for the visit, and the financial obligations of the patient and provider for the specific patient visit.

2. The method of claim 1 and including transmitting through a communications network to an insurance provider its financial obligation for the specific patient's examination, treatment, and visit.

3. The method of claim 1 and including said step of inputting data relative to the patient's examination further including inputting information for recording relating to the physical examination, inputting data relative to the patient's explanation of symptoms of the present illness, recording information relative to a practitioner's diagnosis, studies and tests ordered, and treatments to be given.

4. The method of claim 3 and including said step of calculating the financial obligation further including determining specific charges, payments made, and balance due relative to the patient's examination, treatment and visit.

5. The method of claim 4 and including inputting information relative to any outside office tests conducted, a location of said tests, any medication prescribed, a location of said medication any referral to another specialist for further treatment and a location of the other specialist, and a schedule for further examinations, treatments, and visits of the specific patient.

6. The method of claim 5 wherein the step of storing in memory includes the provision of initiating a save record button for preserving upon a single record of the medical and financial data relative to the specific patient under medical investigation and treatment.

7. A system for creation of an integrated single page medical record via a communications computer network, comprising:
means for recording the scheduling of a patient's visit, means for recording data relative to the patient's clinical visit, including means for recording the history of the patient and entering data relative to the physical examination of the patient, and means for recording data relating to the diagnosis, testing, and treatment planning for the patient, and means for scheduling the sign-out of the patient, and means for recording the establishment of next visit date, and means for scheduling the studies and referrals relating to the patient, all on said single page;
means for recording data relative to the financial information pertaining to the patient's clinical visit, including means for gathering and verifying the patient's insurance coverage information, means for recording data relative to the patient's visit and treatment upon a charge sheet establishing the charges for the patient's visit, means for entering said financial data for the visit into a billing system, means for recording the data into the patient's ledger, listing of the patient's charges, and payments to date, and means for generating a statement to the patient for the balance due from the ledger relative to the current and previous visits all on said single page; and
means for electronically forwarding upon the computer the data relative to the patient's most current visit, transmitting data to an insurance provider, relative to the services conducted for that visit, and means for processing upon the computer the financial statement, payments made thereto, and the amount of patient billings in arrears for collection, all on said single page.

8. A method and system for creation of an integrated medical patient's record all on a single page via a computer, including providing a record of standard patient personal and medical history, and which is capable of receiving data pertaining to a specific visit for the patient being treated;
inputting data relative to the personal history of the specific patient to be examined and treated all on said single page;
inputting data all on said single page relative to specific medical information determined during the specific patient's examination and visit;
inputting data all on said single page relative to the patient's diagnosis, studies, treatment and visit charge during that specific patient visit, and inputting the financial requirements needed for medical coverage by the patient's insurance provider and the patient;
deriving and calculating the financial obligations regarding the patient's diagnosis, studies, treatment, and visit charges for determining the patient and insurance provider financial obligations all on said single page;
providing a single page record for said diagnosis, studies, treatment, and examination, and the financial obligations determined for that patient's specific visit; and
storing electronically in memory the statement of the patient's medical history for the visit, and the financial obligations of the patient and provider for that specific patient visit all on said single page.

9. The method of claim 8 and including inputting at least one code categorizing the charges made relative to the type of study, treatment, and diagnosis provided by a physician for a specific patient visit.

10. The method of claim 9 wherein said providing a single page record for said diagnosis, studies, treatment, and examination, and the financial obligations determined for that patient's specific visit is created in a single step by said method.

11. The method and system of claim 1 and including the recording of patient data onto a single form in the examination room, with the patient being present, and all of the clinical information needed for patient care, consultation, and the billing charges pertinent to that work can be generated immediately from the recorded visit information.
